(19) United States
(12) Patent Application Publication
(10) Pub. No.: US 2011/0098731 A1
(43) Pub. Date: Apr. 28, 2011

(54) MAGNETICALLY ASSISTED CLASPS FOR PROSTHETIC IMPLANTS, AND RELATED METHODS

(76) Inventors: Eric Whitbrook, St. Paul, MN (US); Craig A. Ekvall, East Bethel, MN (US); Jon St. Germain, Elk River, MN (US); Alex Peterson, Maple Grove, MN (US)

(21) Appl. No.: 12/902,207

(22) Filed: Oct. 12, 2010

Related U.S. Application Data
(60) Provisional application No. 61/254,861, filed on Oct. 26, 2009.

(51) Int. Cl. A61B 17/00

(52) U.S. Cl. 606/151

(57) ABSTRACT

A prosthesis for implanting in a patient may include an elongated, laterally flexible structure having first and second end portions that are spaced from one another along a length of the elongated structure. First and second magnetic structures may be respectively secured to the first and second end portions. The first and second magnetic structures may magnetically attract one another when brought into proximity with one another. This magnetic attraction can facilitate achieving desired final relative positioning and/or alignment of the magnetic structures. Thereafter, this magnetic attraction can keep (or help to keep) the end portions of the prosthesis proximate to one another, thereby forming the prosthesis into a desired closed ring shape.

This application claims the benefit of U.S. provisional patent application No. 61/254,861, filed Oct. 26, 2009, which is hereby incorporated by reference herein in its entirety.

### BACKGROUND

The following “background” material is intended to provide some generally helpful context and motivation for this disclosure. Nothing said about magnetism or the like in this “Background” section is admitted by the inventors hereof to be “prior art” against any part of this disclosure.

This disclosure relates to medical implants where the joining of one or more ends or connections can be made easier by employing magnetic attraction. Minimally invasive surgery can be technically difficult, requiring a high degree of skill and coordination. The useable surgical field may be small and the tools may have limited degrees of movement and functionality. Using magnetic attraction to assist the surgeon therefore can be advantageous. An illustrative use of the subject matter of the disclosure is connecting the ends of a medical device during laparoscopic anti-reflux surgery.

During laparoscopic surgery one or more small incisions are made in the abdominal cavity. The abdominal cavity and surgical field is then often further increased in size by insufflation with carbon dioxide. Through these incisions, ports are placed. Cameras and tools are then inserted through these ports and used to operate on the object of interest. Working through small incisions requires special long tools with limited functionality. Using these tools makes traditional surgical techniques such as suturing and knot tying difficult or impossible in some situations. In the case of laparoscopic anti-reflux surgery, a medical implant may be passed around the esophagus at the level of the lower esophageal sphincter, and the two ends of the implant are joined and secured by suture. Either a surgical knot is tied or a suture clip or other similar apparatus may be used. These approaches can be technically difficult, time consuming, and at added cost when an additional device is used in place of a knot. Furthermore, creating a knot that is tight, without slack, and doesn't slip is also technically challenging using laparoscopic instruments. Surgeons are continually trending towards less invasive approaches. For laparoscopic surgery this means smaller ports, fewer ports (single port procedures are being performed), and ports placed in “natural orifices” or so-called N.O.T.E.S. procedures (Natural Orifice Transluminal Endoscopic Surgery). Magnetically aided connections will become increasingly important as fewer tools are used and smaller spaces are entered.

### SUMMARY

The present disclosure aims to address the above and other possible drawbacks of implanting a device by minimally invasive laparoscopic surgery. Certain aspects of the disclosure may also have other applications, so the disclosure is not wholly confined to laparoscopic anti-reflux surgery. For instance, this disclosure may be useful for securing a device during minimally invasive obesity surgery or surgery for fecal or urinary incontinence. However, anti-reflux surgery will be mentioned most frequently herein and it serves as a useful context in which to illustrate the disclosure.

During one type of anti-reflux therapy, a bracelet-like device is wrapped around the esophagus and the two ends of the initially open bracelet are brought together and secured. In this application it is essential that once the patient is recovered back to normal eating patterns, the device does not detach during normal physiological events such as swallowing or vomiting. At the level of the device, the pressure inside the esophagus can be anywhere from 5-50 mmHg during swallowing and as high as 200-250 mmHg during vomiting. These physiological events can therefore place significant mechanical loads on the device. In some instances the attractive force of one or more permanent magnets may be enough to secure a connection and withstand all relevant physiological loads. However, it may be undesirable to use a large enough magnet or magnets to effect a sufficient attractive force. As in the case of anti-reflux surgery, the device must be sufficiently small to pass through the ports (usually 5-15 mm inner diameter) and be able to be placed around the esophagus. In such applications it is advantageous to use magnetic attraction to facilitate bringing the ends of a device together, as well as a secondary mechanical interengagement and/or lock between the ends to be able to withstand the physiological loading conditions. The magnetic connection can be reversible as in the examples in accompanying FIGS. 1-7 and 31-33, or permanent as in the examples in accompanying FIGS. 8, 9 and 27-30.

In some instances of laparoscopic surgery it may be desirable to connect two ends of a device, collect information or measurements, disconnect the device, and reposition or make other changes. In other instances it is desirable for the surgeon to be unable to reverse the connection, e.g., to reduce user error or accidental disconnection.

In accordance with certain possible features of the disclosure, apparatus (e.g., a prosthesis) for implanting in a patient's body may include an elongated, laterally flexible structure having first and second end portions that are spaced from one another along a length of the elongated structure. First and second magnetic structures may be secured to the first and second end portions, respectively. When brought into proximity with one another, the first and second magnetic structures may magnetically attract one another. This magnetic attraction can help complete final alignment and positioning of the magnetic structures relative to one another. Thereafter, this magnetic attraction can keep (or help to keep) the magnetic structures together. This, in turn, keeps the first and second end portions of the prosthetic apparatus proximate to one another. For example, this may give the prosthetic apparatus a desired closed ring configuration around a tissue structure (such as the esophagus) in the patient's body.

In accordance with certain of its other possible features, the disclosure may relate to implanting in a patient's body apparatus (e.g., a prosthesis) that includes (1) an elongated, laterally flexible structure having first and second end portions that are spaced from one another along a length of the elongated structure, and (2) first and second magnetic structures that are respectively secured to the first and second end portions. The method may include engaging each of the first and second magnetic structures (e.g., via respective first and second laparoscopic instruments) and manipulating those structures (e.g., at least partly inside the patient's body) to bring those structures into proximity with one another so that magnetic attraction between those structures can help them achieve final desired positions relative to one another. Thereafter, that magnetic attraction may keep (or help to keep) the magnetic structures in their desired relative positions, which, in turn, may maintain the first and second end portions of the prosthetic apparatus proximate to one another as desired for the final, implanted condition of the prosthesis. Again, this final implanted condition may be one in which the prosthesis forms a ring that is closed around a tissue structure (such as the esophagus) inside the patient's body.

Further features of the disclosure, its nature and various advantages will be more apparent from the accompanying drawings and the following detailed description.

### BRIEF DESCRIPTION OF THE DRAWINGS

FIG. 1 is a simplified isometric or perspective view of an illustrative embodiment of apparatus in accordance with certain possible aspects of the disclosure.

FIG. 2 is a simplified isometric or perspective view of another illustrative embodiment of apparatus in accordance with certain possible aspects of the disclosure.

FIG. 3 is a simplified isometric or perspective view, at least partly in section, of still another illustrative embodiment of apparatus in accordance with certain possible aspects of the disclosure.

FIG. 4 is a simplified isometric or perspective view, at least partly in section, of yet another illustrative embodiment of apparatus in accordance with certain possible aspects of the disclosure.

FIG. 5 is a simplified isometric or perspective view, at least partly in section, of still another illustrative embodiment of apparatus in accordance with certain possible aspects of the disclosure.

FIG. 6 is a simplified isometric or perspective view, at least partly in section, of yet another illustrative embodiment of apparatus in accordance with certain possible aspects of the disclosure.

FIG. 7 is a simplified isometric or perspective view, at least partly in section, of still another illustrative embodiment of apparatus in accordance with certain possible aspects of the disclosure.

FIG. 8 is a simplified sectional view of another illustrative embodiment of apparatus in accordance with certain possible aspects of the disclosure.

FIG. 9 is a simplified isometric or perspective view, at least partly in section, of still another illustrative embodiment of apparatus in accordance with certain possible aspects of the disclosure.

FIG. 10 is a simplified isometric or perspective view of an illustrative embodiment of apparatus in accordance with this disclosure implanted in an illustrative manner in a patient's body, also in accordance with this disclosure.

FIG. 11 is a simplified depiction of portions of a patient's body anatomy with illustrative laparoscopic instrumentation being used on the patient's body.

FIG. 12 is similar to FIG. 10 prior to implantation of apparatus in accordance with this disclosure.

FIGS. 13-21 are each similar to FIGS. 10 and 12, but show successive stages (in accordance with certain possible aspects of this disclosure) in implanting illustrative apparatus in accordance with disclosure in a patient's body.

FIG. 22 is an enlargement of a portion of FIG. 21.

FIG. 23 is another depiction of a portion of a patient's internal anatomy or body tissue structures.

FIG. 24 is similar to FIG. 23 but with an illustrative prosthesis in accordance with certain aspects of the disclosure implanted in the patient.

FIG. 25 is generally similar to FIG. 24 but shows the condition of the prosthesis during a certain physiological condition of the patient's tissue. Some aspects of FIG. 25 are shown in section.

FIG. 26 is similar to FIG. 25 but shows the condition of the prosthesis during a certain different physiological condition of the patient's tissue.

FIG. 27 is a simplified elevational view of another illustrative embodiment of apparatus in accordance with certain possible aspects of the disclosure. Some portions of FIG. 27 are shown as transparent or translucent to reveal interior structures.

FIG. 28 is similar to FIG. 27 but shows a different operating condition of the FIG. 27 apparatus in accordance with certain possible aspects of the disclosure. Again, some aspects of FIG. 28 are shown as transparent or translucent to reveal interior structures.

FIG. 29 is a simplified isometric or perspective view of one of the components or subassemblies of the apparatus that is shown in FIGS. 27 and 28.

FIG. 30 is similar to FIG. 29 but shows another illustrative embodiment of how what is shown in FIGS. 27-29 may be constructed in accordance with certain possible aspects of the disclosure.

FIG. 31 is a simplified isometric or perspective view of another illustrative embodiment of apparatus in accordance with certain possible aspects of the disclosure.

FIG. 32 is similar to FIG. 31 but shows another operating condition of the FIG. 31 apparatus in accordance with certain possible aspects of the disclosure.

FIG. 33 is a simplified isometric or perspective view of one of the components of the apparatus shown in FIGS. 31 and 32.

### DETAILED DESCRIPTION

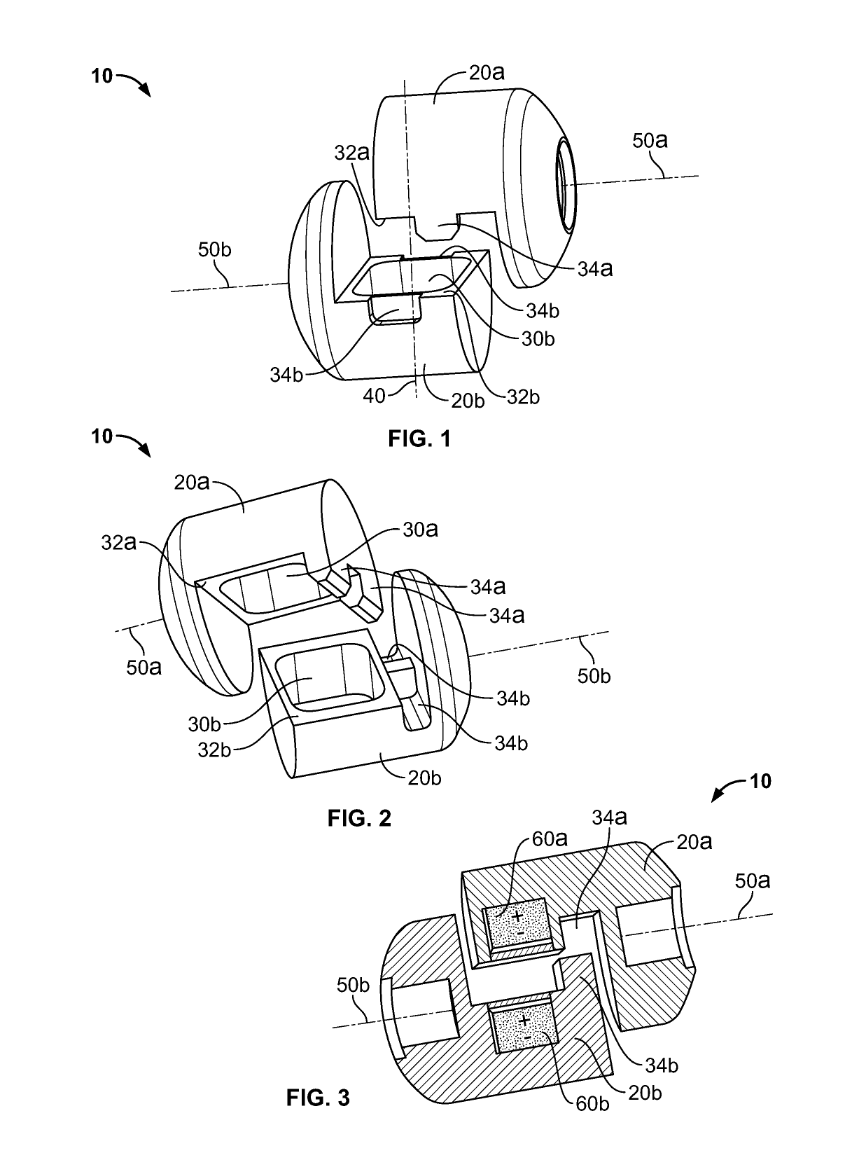

In the illustrative embodiment shown in FIG. 1, clasp 10 includes first clasp structure 20a and second clasp structure 20b. Each of these clasp structures is attached to a respective opposite end of an implantable prosthetic “bracelet”, e.g., of any of the types shown in Kugler et al. U.S. patent application publication 2005/0283235 or Berg et al. U.S. patent application publication 2009/0062824, both of which are hereby incorporated by reference herein in their entireties. The purpose of clasp 10 and all of the other clasp structures shown herein is to facilitate connecting together the two opposite ends of such a bracelet during implanting of the bracelet in a patient, and to at least help to secure those two bracelet ends together after the two parts of the clasp structure have been joined in the patient. In other words, the two parts (e.g., 20a and 20b) of such a clasp 10 are initially separate as the bracelet is introduced into the patient. But at the appropriate time during the implanting procedure, the two parts of the clasp are brought toward one another so that they can join together and hold the bracelet closed around a body tissue structure in the patient. To facilitate such joining of the two clasp parts at a location in the patient that may be relatively inaccessible to the surgeon or other user, and/or that may be accessed only by means of instruments that are somewhat limited in their manipulative ability, the clasp structures of this disclosure preferably include magnetic elements for magnetically attracting the two parts of a clasp toward one another and ultimately into engagement with one another.

Returning to FIG. 1, each part 20a and 20b of clasp 10 includes a recess 30a (not shown) or 30b, which can receive and permanently retain a permanent magnet or magnetic element (not shown) that is magnetically attracted to the magnet or magnetic element in the other clasp part. At least one of these two magnetic elements must be an actively magnetic element like a permanent magnet. The other magnetic element can also be a permanent magnet, or alternatively it can be a passively magnetic element such as a piece of ferromagnetic material that is magnetically attracted to an actively magnetic permanent magnet. For simplicity of discussion, all such elements will typically be referred to simply as magnets or magnetic elements, it being understood that in any pair of such elements both may be actively magnetic, or one may be actively magnetic while the other is only passively magnetic.

Returning again to FIG. 1, the line of magnetic attraction between clasp parts 20a and 20b is parallel to the imaginary or geometrical axis 40 in FIG. 1. Axis 40 is perpendicular to the longitudinal axes 50a and 50b of the end portions (not shown) of the bracelet that are attached to clasp parts 20a and 20b, respectively. Axis 40 is also perpendicular to the face 32a/32b of the structure of the clasp part that surrounds the recess 30a/30b containing the magnetic element of each clasp part. Accordingly, once clasp parts 20a and 20b are relatively close to one another as shown in FIG. 1, the magnetic attraction between those clasps parts pulls them toward one another along axis 40 until the faces 32a and 32b on the respective clasp parts came into contact with one another.

When surfaces 32a and 32b contact one another as described above, a projection 34a on each side of clasp part 20a enters a respective recess 34b on each side of clasp part 20b. This insertion of elements 34a into elements 34b (parallel to axis 40) acts as a mechanical preventative against clasp parts 20a and 20b pulling apart or moving away from one another in a direction parallel to axes 50a and 50b. In other words, the patient's body tissue structure around which the bracelet has been implanted may, from time to time, put tension on clasp 10 parallel to axes 50a/50b. But clasp 10 is able to resist this tension and avoid pulling apart because projections 34a are in recesses 34b. The magnetic attraction between parts 20a and 20b helps to keep projections 34a in recesses 34b, and this magnetic attraction may also help prevent clasp parts 20a and 20b from pulling apart along axes 50a and 50b. But in this embodiment, the primary resistance to pulling apart may be the mechanical interfitting of elements 34a and 34b, and the primary function of the magnetic attraction after parts 20a and 20b have initially come together may be to keep elements 34a in elements 34b so that those latter elements can provide secure mechanical resistance of parts 20a and 20b to pulling apart parallel to axes 50a and 50b.

Another illustrative embodiment is shown in FIG. 2. Although this embodiment differs in some respects from FIG. 1, the same reference numbers are used again in FIG. 2 for generally similar elements. Thus the description of FIG. 2 can be somewhat briefer. In FIG. 2 clasp part 20a again has a recess 30a for a magnet (not shown). Clasp part 20b also has a recess 30b for a magnet (not shown). These two magnets magnetically attract one another along an axis (similar to axis 40 in FIG. 1) that is perpendicular to the surfaces 32a and 32b that surround recesses 30a and 30b. When clasp parts 20a and 20b come together along this axis, projections 34a, which project from surface 32a of part 20a, extend into recesses 34b in part 20b. (Recesses 34b extend below surface 32b.) This interengagement of features 34a and 34b provides mechanical resistance to (indeed, actual mechanical prevention of) clasp parts 20a and 20b pulling apart along axes 50a and 50b. Again, the magnetic attraction between parts 20a and 20b keeps elements 34a and 34b mechanically interengaged, while that mechanical interengagement may be what primarily prevents parts 20a and 20b from pulling apart along axes 50a/b.

FIG. 3 shows another embodiment that is somewhat like the FIG. 2 embodiment. Again, the same reference numbers are used for generally similar features. FIG. 3 actually shows the magnetic element 60a or 60b in each clasp part 20a/20b, respectively. The magnetic polarities of magnetic elements 60a and 60b are indicated in FIG. 3 (and other FIGS.) by the plus (“+”) and minus (“−”) signs drawn on these elements. In all cases the “+” pole of one of these magnetic elements 60 is oriented to face toward the “−” pole of the other one of these elements in any pair of such elements. There is magnetic attraction between these oppositely polarized ends or poles of two magnetic elements 60 that thus face toward one another. FIG. 3 further shows that each magnetic element 60 can be fully embedded in the other material of the respective clasp part, if desired. Further description of FIG. 3 should not be needed because of its otherwise similarity to previously described embodiments.

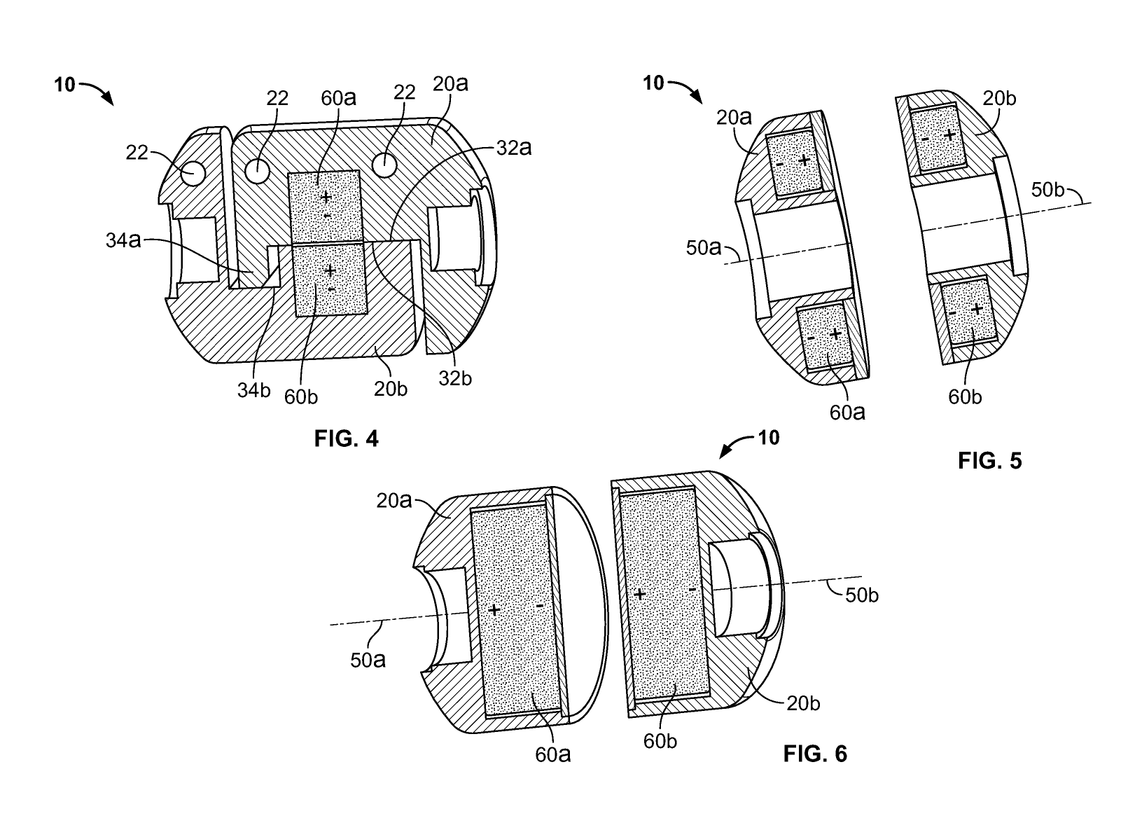

FIG. 4 shows an embodiment that is generally similar to the FIGS. 2-3 embodiments. FIG. 4 again uses the same reference numbers for similar elements. FIG. 4 shows the two parts of clasp 10 actually together. This is the final, finished, implanted condition of clasp 10. Except for the following discussion of holes 22, further description of FIG. 4 should not be needed.

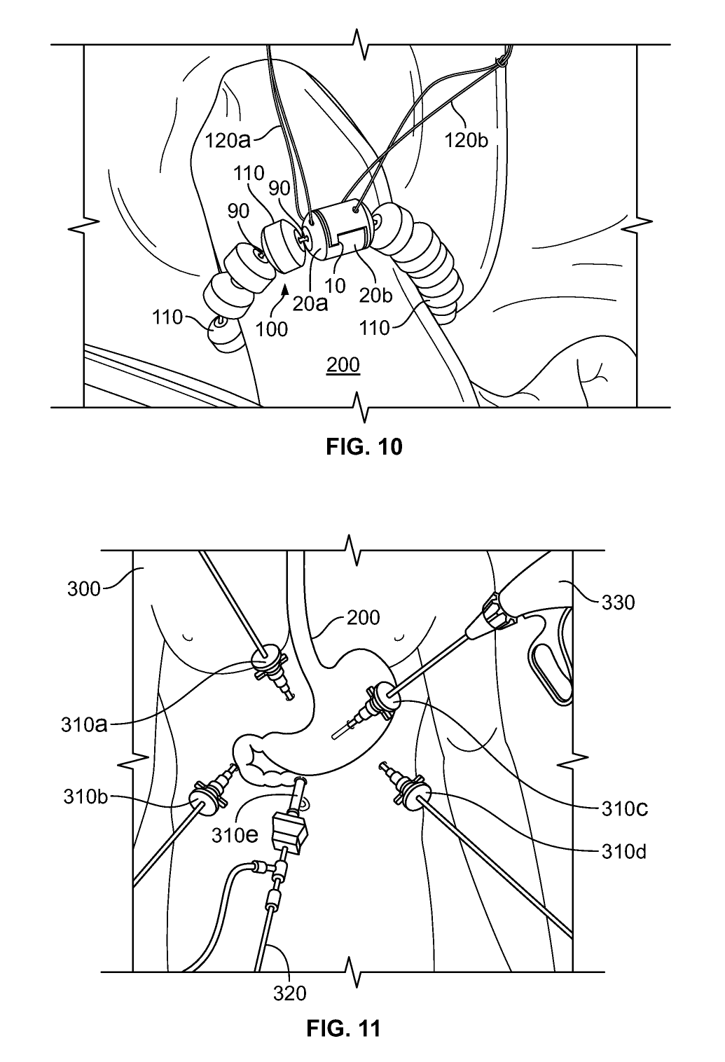

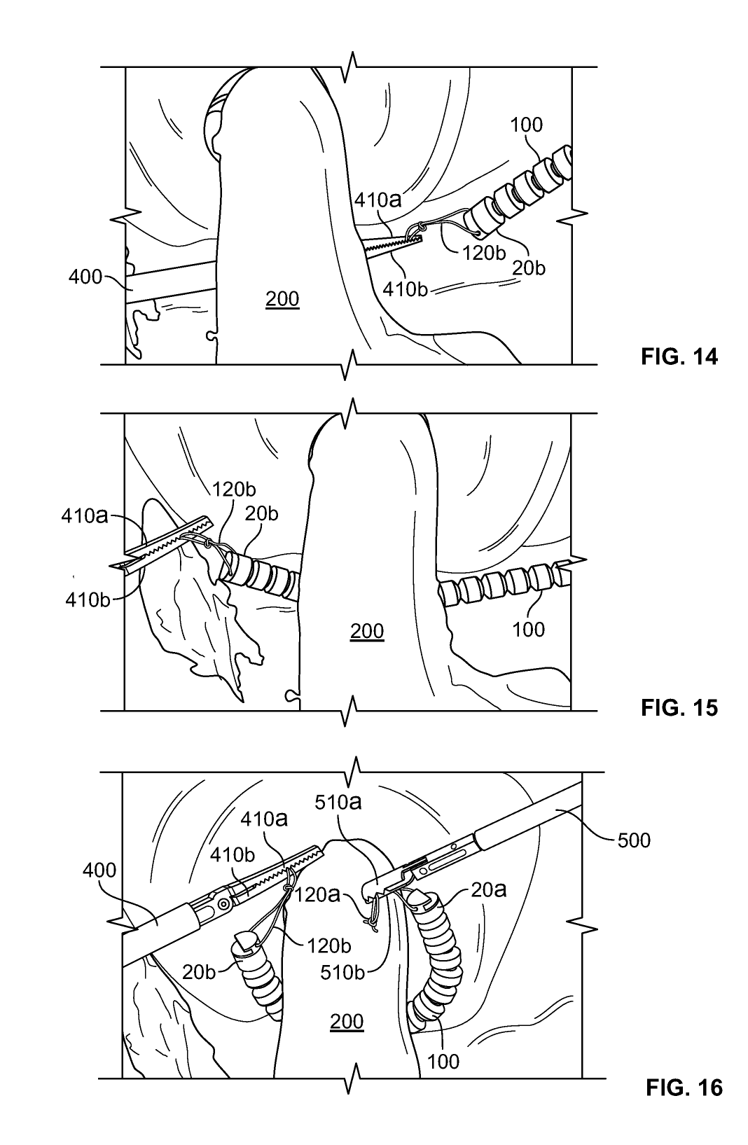

FIG. 4 illustrates the possibility that at least one hole 22 may be provided through each part 20a/20b of a clasp 10. A suture loop may be passed through each such hole 22 (see, for example, suture loops 120a and 120b in FIG. 10). Such suture loops can facilitate manipulation of the clasp parts by laparoscopic tools or the like. Such tools may be somewhat ferromagnetic. It can be helpful to have suture loops for such tools to grasp to reduce the possibility of a clasp part becoming magnetically “stuck” to the tool. FIGS. 14-20 show examples of laparoscopic tools 400 and 500 manipulating the parts 20a and 20b of a clasp by grasping suture loops 120a and 120b rather than clasp parts 20a and 20b themselves. Holes like 22 and suture loops like 120 can be provided in and used with any of the clasp parts 20 shown herein.

FIG. 5 shows an alternative embodiment in which clasp parts 20a and 20b are held together solely by the magnetic attraction between the magnets 60a and 60b in those respective clasp parts. In this case the axis of magnetic attraction between clasp parts 20a and 20b is parallel to the axes 50a/b that lead off to the end portions of the bracelet. In the embodiment shown in FIG. 5, each of magnets 60 is a ring magnet that is substantially concentric about the associated axis 50. The alternative embodiment shown in FIG. 6 is similar, except that in FIG. 6 each magnet 60 is a disc magnet.

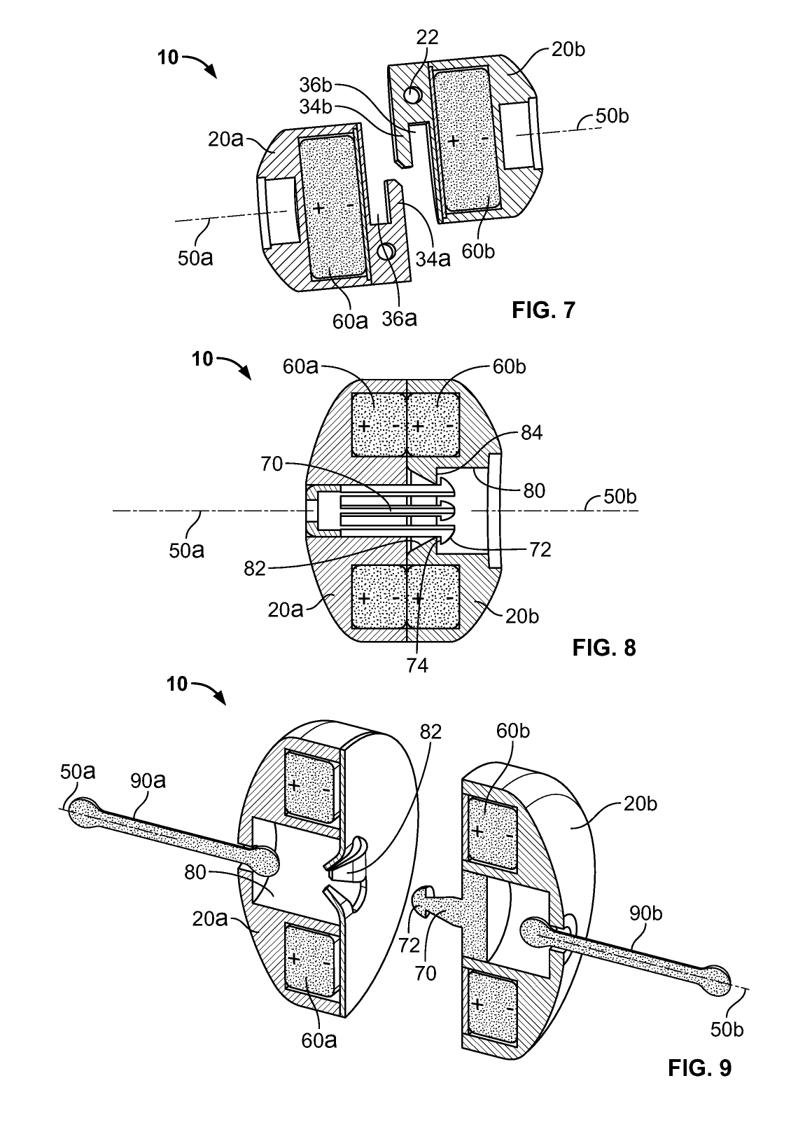

FIG. 7 shows another illustrative embodiment that is similar to the FIG. 6 embodiment, but with the addition of a “knuckle” coupling between clasp parts 20a and 20b. In FIG. 7 clasp parts 20a and 20b are magnetically attracted to one another, generally along a magnetic attraction axis that is parallel to axes 50a and 50b. When clasp parts 20a and 20b are in or near face-to-face contact with one another, they can be shifted relative to one another transverse to axes 50a and 50b so that transverse projection 34a on part 20a extends into transverse recess 36b on part 20b, and transverse projection 34b on part 20b similarly extends into transverse recess 36a on part 20a. The resulting knuckle coupling between elements 34 and 36 provides mechanical assurance that clasp parts 20a and 20b cannot pull apart parallel to axes 50a/b after those parts have been put together.

FIG. 8 shows another embodiment that is somewhat like FIG. 5, except that in FIG. 8 clasp parts 20a and 20b have interengaging latch components that permanently prevent parts 20a and 20b from coming apart in any direction after those parts have been put together. In particular, part 20a includes a plurality of fingers 70 (in an annular array) that extend from part 20a toward part 20b parallel to axes 50. Part 20b has an aperture 80 that opens toward part 20a. Fingers 70 can extend into aperture 80. Cooperating cam surfaces 72 on fingers 70 and 82 on aperture 80 cause the free ends of fingers 70 to resiliently deflect toward one another as surfaces 72 and 82 pass one another. After surfaces 72 and 82 have completely passed one another, fingers 70 spring out resiliently again so that latch surfaces 74 on the fingers engage with latch surface 84 on aperture 80 as shown in FIG. 8. In this final condition of the clasp, cooperation between latch surfaces 74 and 84 permanently mechanically prevents clasp parts 20a and 20b from coming apart after they have been put together. Whereas cam surfaces 72 and 82 are inclined relative to axes 50a/b, latch surfaces 74 and 84 are preferably substantially perpendicular to axes 50a/b. Embodiments like those shown in FIGS. 1-7 can be taken apart. But embodiments like the one shown in FIG. 8 form a more permanent connection between clasp parts 20a and 20b.

FIG. 9 shows another embodiment similar to FIG. 8. FIG. 9 has a different form of permanent mechanical connection between clasp parts 20a and 20b. In FIG. 9 clasp part 20a has a central aperture containing a plurality of fingers that incline inwardly and away from clasp part 20b. Clasp part 20b has a bayonet post 70 that points toward clasp part 20a. Post 70 can enter aperture 80, and the enlarged head 72 on the free end of that post can resiliently push apart the free ends of fingers 82 in the aperture. After the enlarged head 72 of post 70 has passed the free ends of fingers 82, fingers 82 spring back in behind the enlarged head 72 of post 70. This permanently mechanically latches clasp parts 20a and 20b together. Note that FIG. 9 also shows the first (end) links 90a and 90b leading off from clasp parts 20a and 20b to the rest of the bracelet that includes clasp 10.

FIG. 10 shows an illustrative embodiment of a bracelet 100 in accordance with this invention implanted around a patient's body tissue structure 200 inside the patient's body. In this case tissue structure 200 is the patient's esophagus, but it will be understood that tissue structure 200 could instead be any other suitable body tissue structure (e.g., the stomach, the rectum, the urethra, etc.). In this embodiment bracelet 100 includes clasp 10 and a plurality of other bracelet beads 110 that are maintained in an ordered array by links 90 between adjacent elements 10 and 110 in the array. As shown and described in the references that are mentioned above, links 90 typically allow limited movement of adjacent elements 10/110 toward and away from one another along the length of the links. (Note that in this context all references to element 10 are references to that element as a whole, not to its individual subassemblies 20a and 20b.) This allows bracelet 100 to annularly (circumferentially) enlarge or contract (e.g., in response to physiological activity in the surrounded tissue structure—an example being passage of food down the esophagus during swallowing). Circumferentially adjacent ones of elements 10/100 are typically resiliently attracted to one another (e.g., by magnetic attraction). But this magnetic attraction can be overcome by appropriate physiological activity of tissue structure 200. Thus this physiological activity can cause bracelet 100 to annularly enlarge. But after completion of the physiological activity, the attraction between adjacent elements 10/110 causes the bracelet to automatically return to its earlier, circumferentially smaller condition.

FIG. 10 also shows the inclusion of two manipulation aids 120a and 120b on clasp parts 20a and 20b, respectively. In this embodiment manipulation aids 120 are strands or loops of flexible material such as suture thread. Several of the earlier FIGS. show apertures 22 in clasp parts 20 through which such manipulation aids 120 can be looped.

If possible, it is typically desired to implant a bracelet like 100 in a patient's body using a procedure that is less invasive than full, open-body surgery. Such less invasive procedures will be referred to herein as minimally invasive or laparoscopic, although it will be understood that the degree of invasiveness may vary up to but typically not including full open-body (or traditional) surgery.

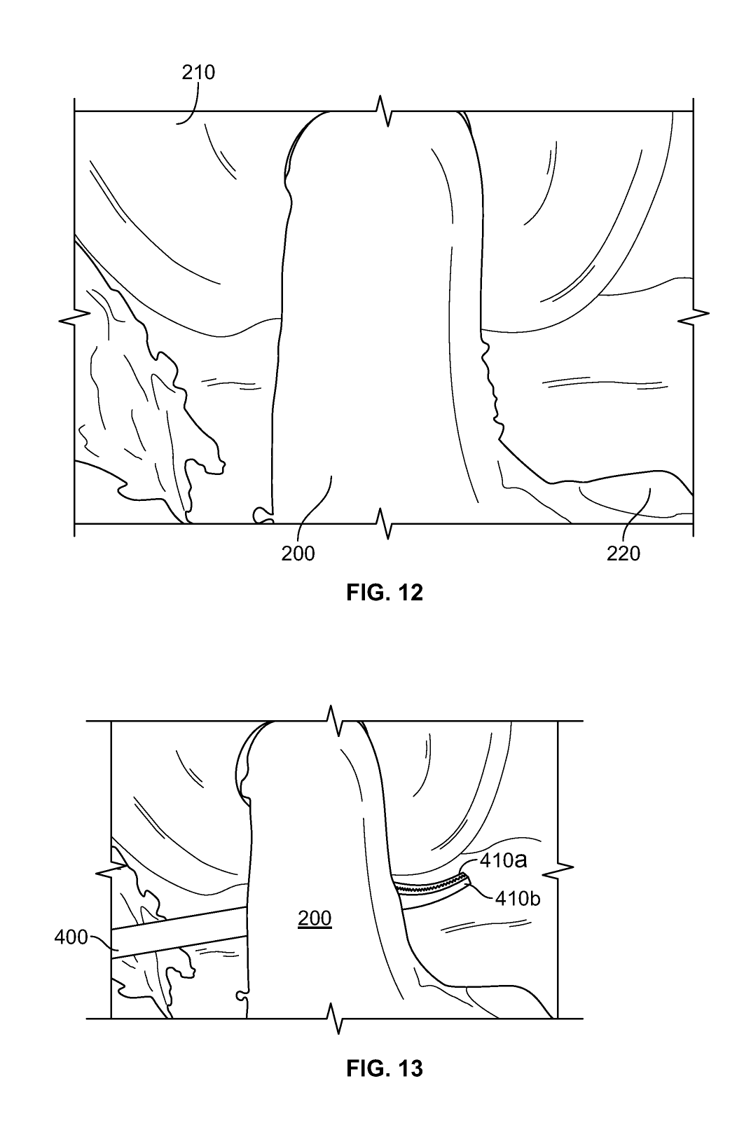

FIG. 11 shows some examples of approaches that can be used for minimally invasive implantation of a bracelet like 100. The patient's body is indicated in part by reference number 300. The locations of certain body structures inside the patient are also indicated in FIG. 11 (although those internal body structures are not in fact visible from the outside of the patient's body). FIG. 11 shows examples of several locations where relatively small instrument entry ports 310a-e can be placed through the skin, fascia, and other relatively superficial tissues to gain access to the interior of the patient's body. FIG. 11 shows a laparoscope (camera) 320 going in through one of these entry ports. FIG. 11 shows a clip applier 330 going in through another of these entry ports. Any of these (or other) entry ports 310 can be used to introduce into the patient's body bracelet 100 and instruments for manipulating the bracelet inside the body to close clasp 10 and complete the implantation of the bracelet around a body tissue structure such as the esophagus 200 in the patient. FIG. 12 begins a series of FIGS. that shows this being done in a minimally invasive way (e.g., through entry ports like 310 in FIG. 11). It will be understood that the instruments shown in these FIGS. are manipulated and controlled from outside the patient's body. Thus control of what goes on at the distal ends of these instruments is effectively “remote control.” There is no direct access to these distal ends, and so it is important to facilitate operations that must take place at those remote and relatively inaccessible locations.

FIG. 12 shows the esophagus 200 and some adjacent tissue structures prior to approach by bracelet 100 and the instrumentation for implanting the bracelet. For example, in addition to esophagus 200, FIG. 12 shows the patient's diaphragm 210 above the bracelet implant site, and the patient's stomach 220 below the bracelet implant site.

FIG. 13 shows the distal end portion of a first elongated, minimally invasive instrument 400 being introduced into the patient (e.g., via an entry port 310) so that the distal end portion of that instrument passes around behind esophagus 200. The distal end of instrument 400 includes a pair of openable and closeable jaws 410a and 410b. Jaws 410 can open to receive manipulation aid 120b on bracelet 100, and to then close on and hold onto that manipulation aid. FIG. 14 shows instrument 400 after its jaws 410 have thus gripped manipulation aid 120b on one end of bracelet 100 (the bracelet having begun introduction into the patient in a linear (i.e., clasp 10 open) condition via another entry port 310). FIG. 14 also shows instrument 400 being used to begin to pull bracelet 100 around behind esophagus 200.

FIG. 15 shows instrument 400 having pulled one open end of bracelet 100 all the way past the rear of esophagus 200.

FIG. 16 shows another instrument 500 (similar to instrument 400, and introduced into the patient through another entry port 310 similarly to instrument 400) gripping the manipulation aid 120a on the other open end of bracelet 100. Like instrument 400, instrument 500 has openable and closeable jaws 510a/b for selectively (remotely controllably) gripping or releasing manipulation aid 120a.

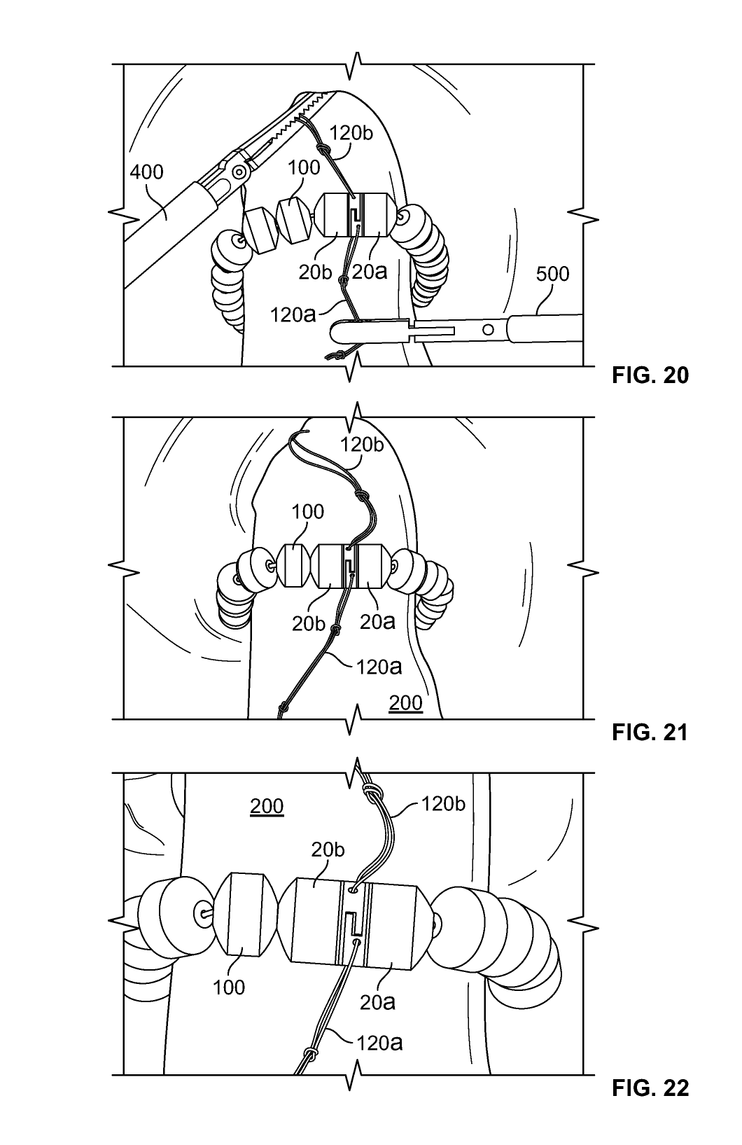

The sequence of FIGS. 16, 17, and 18 shows instruments 400 and 500 being used to gradually move clasp parts 20a and 20b into proximity to one another at or near the front of the esophagus. When clasp parts 20a and 20b reach sufficiently close proximity to one another, the magnetic attraction between those parts begins to aid in completing the proper final alignment and positioning of those parts relative to one another. This can be seen, for example, in FIG. 19, where the magnetic attraction between clasp parts 20a and 20b has helped to bring their respective axes 50a and 50b into parallelism with one another, and has also helped to bring facing surfaces of those clasp parts into contact with another. In FIG. 20 clasp parts 20a and 20b have been further urged into the condition in which their axes 50a and 50b are coaxial, and any mechanical interengagement between parts 20a and 20b is completed.

FIG. 21 shows the completely implanted bracelet 100 after removal of instruments 400 and 500. FIG. 22 is a close-up of the clasp portion 10 of what is shown in FIG. 21.

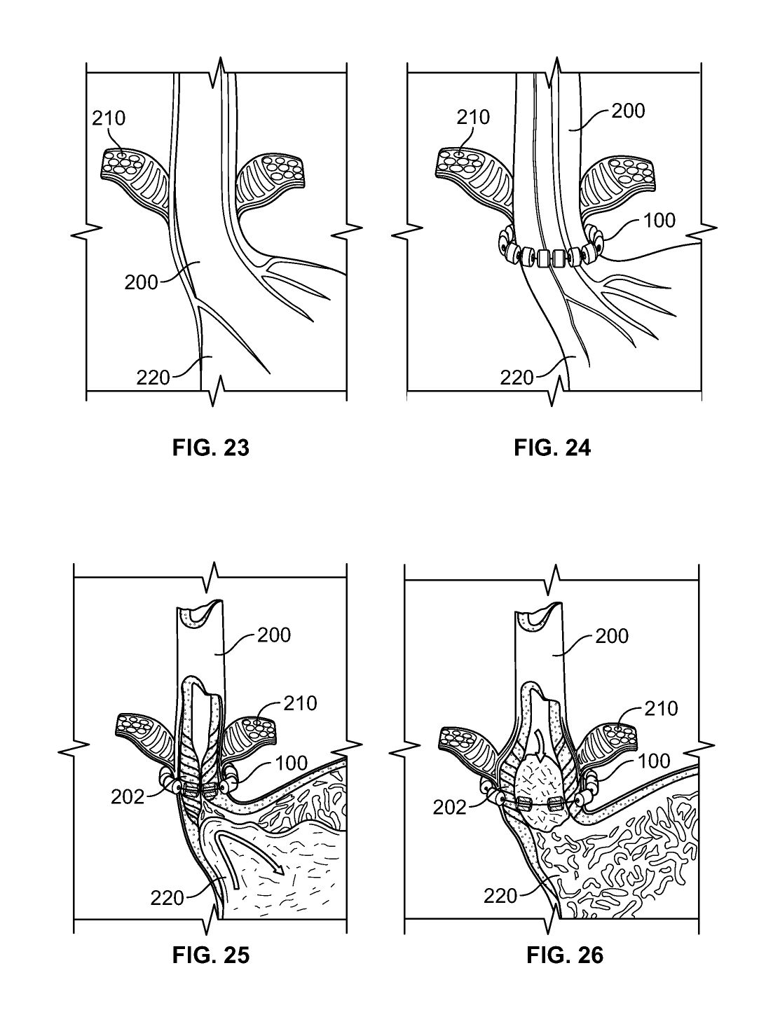

For completeness, FIG. 23 shows the above-described patient anatomy prior to implanting bracelet 100. FIG. 24 shows the FIG. 23 anatomy after implanting bracelet 100. FIG. 25 shows the effect of bracelet 100 in resisting undesirable reflux of the contents of stomach 220 into the esophagus 200 by helping to keep the lower esophageal sphincter 202 closed. FIG. 26 shows how bracelet 100 can temporarily enlarge circumferentially to allow a bolus of swallowed food down through the lower esophageal sphincter 202 and into the stomach.

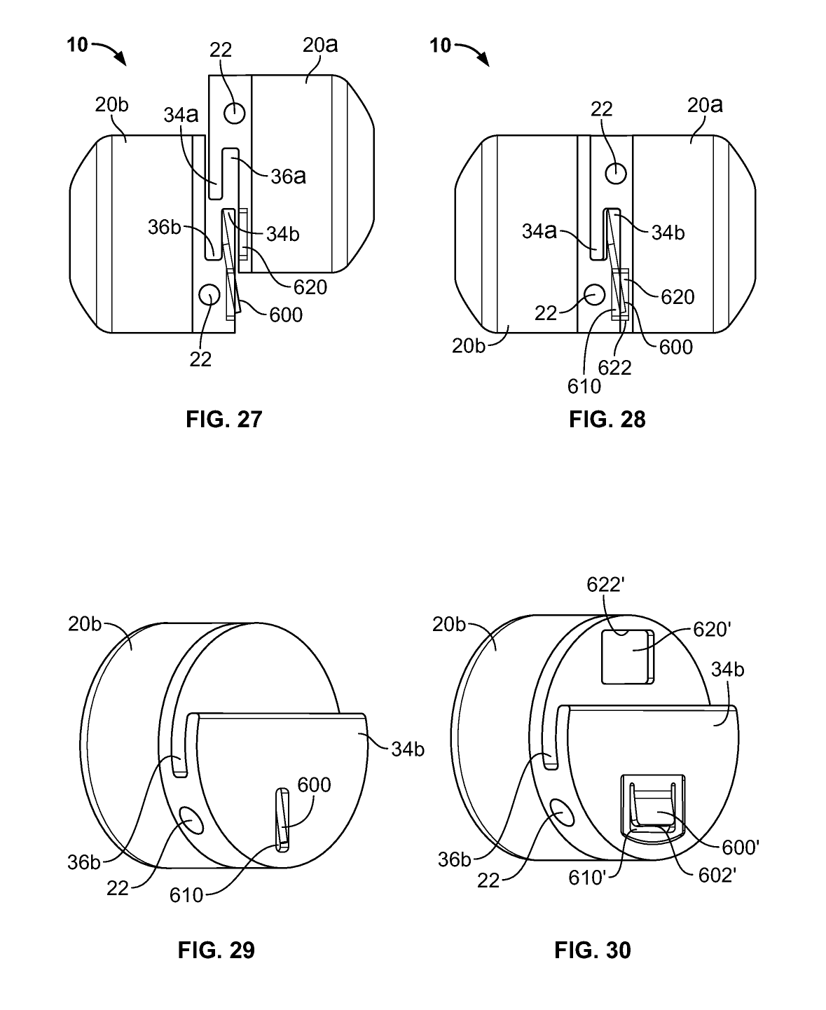

FIGS. 27-29 show another illustrative embodiment of a clasp 10. In respects other than those discussed specifically below, this embodiment can be similar to the embodiment shown in FIG. 7. What the FIGS. 27-29 embodiment adds to the FIG. 7 embodiment is a resilient wire 600 that is secured to clasp part 20b. A cantilevered, free-end portion of wire 600 extends out of a recess 610 in clasp part 20b toward clasp part 20a as clasp parts 20a and 20b are brought together as shown in FIG. 27. This cantilevered portion of wire 600 is inclined to diverge away from the rest of clasp part 20b in the direction that clasp part 20a must move relative to clasp part 20b in order for the clasp parts to interengage with one another. As such interengagement takes place, clasp part 20a initially deflects the free-end portion of wire 600 back into recess 610. Eventually, however, the free-end portion of wire 600 can again spring out into a recess 620 in the adjacent face of clasp part 20a (see FIG. 28). When this happens, the interengagement of clasp parts 20a and 20b is made permanent as a result of the extreme free end of wire 600 interfering with the adjacent end 622 of recess 620. Only if wire 600 or other components of the device are broken can clasp parts 20a and 20b be separated from one another again.

The alternative embodiment that is shown in part in FIG. 30 is similar to the embodiment of FIGS. 27-29, except that in the FIG. 30 embodiment wire 600 is replaced by a resilient tab 600′. When the two parts of a FIG. 30 clasp are interengaged, the free end portion of tab 600′ extends out of recess 610′ into a recess 620′ in the other clasp part. The extreme free end 602′ of tab 600′ is adjacent an end 622′ of slot 620′, and these two features (602′ and 622′) interfere with one another to prevent disengagement of the two parts of the clasp. Only by breaking one or more features of the clasp can the two clasp parts be separated after they have interengaged.

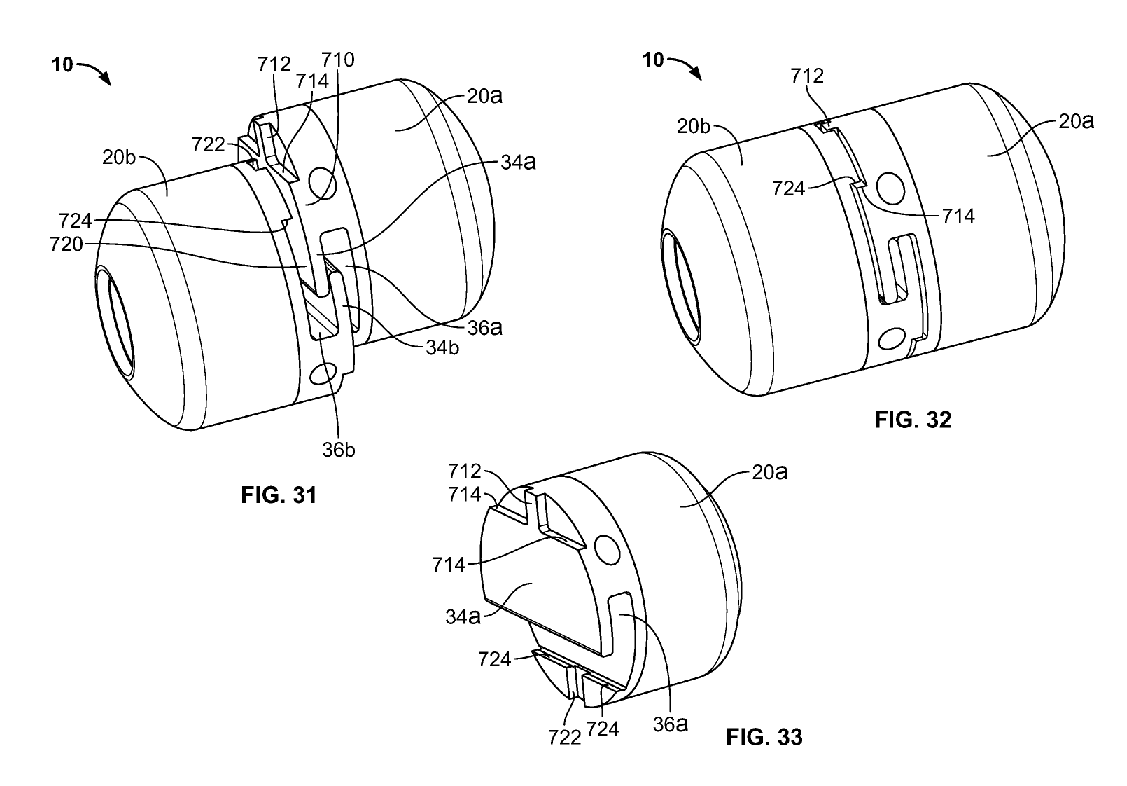

FIGS. 31-33 show yet another illustrative embodiment of a clasp 10. Once again, this embodiment is generally similar to the embodiment shown in FIG. 7, except for the added features that are discussed specifically below. The face of each clasp part 20a or 20b that faces the other clasp part includes a more outwardly projecting portion 34a or 34b on one side of that face, and a more recessed portion 36a or 36b on the other side of that face. The more outwardly projecting portion 34a/b includes a pair of ledges 714 that face away from the side (36a/b) of the face that is more recessed. Between those ledges 714 the more outwardly projecting portion 34a/b continues as a bead 712. The more recessed portion 36a/b has a pair of outwardly projecting ledges 724 that face toward the side (34a/b) of the face that is more outwardly projecting. Between these ledges 724 is a slot 722.

When the two parts 20a and 20b of the clasp 10 in FIGS. 31-33 are fully engaged (see especially FIG. 32), the bead 712 on each part 20 fits in the adjacent slot 722 in the other part 20. In addition, the ledges 714 on each part 20 face adjacent ledges 724 on the other part 20. These various surface features can get to this condition because there is enough clearance for each portion 34a/b to enter the portion 36a/b of the other part. But once this condition is reached, the magnetic attraction between parts 20a and 20b keeps beads 712 in slots 722, and keeps ledges 714 facing ledges 724. These interfering surface features maintain parts 20a and 20b in their relative positions as shown in FIG. 32 in all three dimensions. However, the interengagement of parts 20a and 20b can be deliberately undone, if desired, as follows. Parts 20a and 20b can be pulled apart with enough force to overcome the magnetic attraction between them. This pulls each bead 712 out of the adjacent slot 722, and also pulls ledges 714 away from facing ledges 724. Then parts 20a and 20b can be separated from one another by movement back through the condition shown in FIG. 31, ultimately leading to complete separation of parts 20a and 20b.

From the foregoing it will be seen that while the parts 20a and 20b are fully interengaged as shown in FIG. 32, the magnetic attraction between those parts keeps surface features on those parts adjacent to one another in such a way that there is three-dimensional mechanical stability between those parts. That stability can be deliberately overcome by overcoming the magnetic attraction force and separating parts 20a and 20b by relative movement opposite to the relative movement used to bring those parts together.

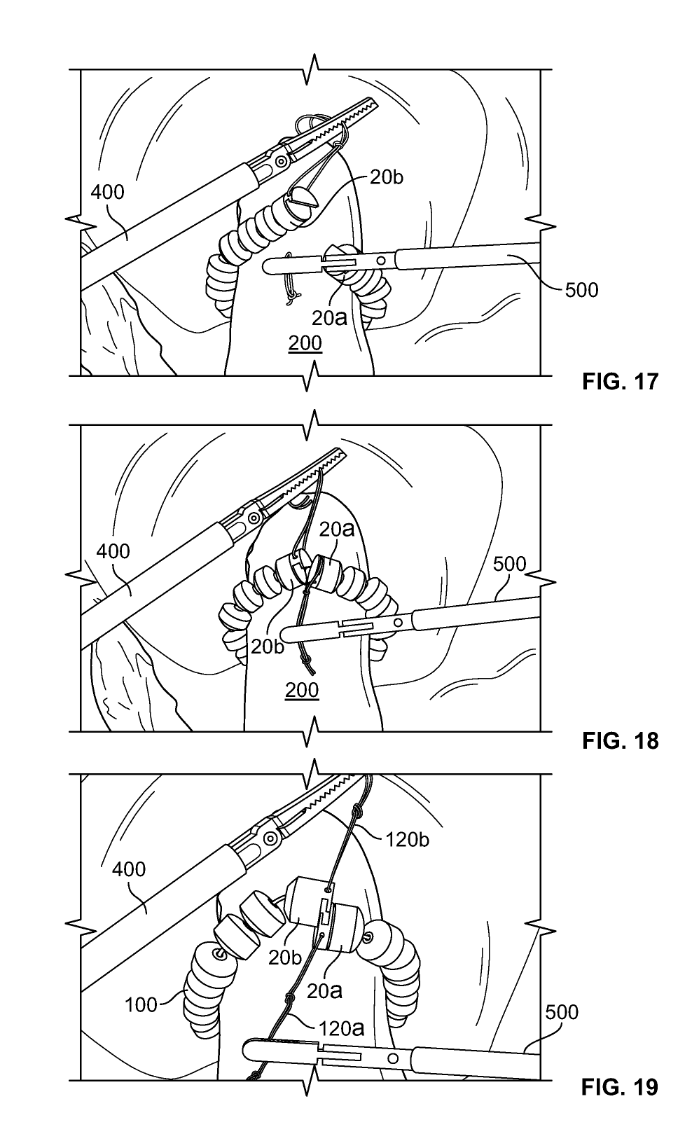

In some respects recapitulating and/or extending the foregoing, certain aspects of the disclosure relate to apparatus (e.g., 10, 100) for implanting in a patient's body (e.g., 300). The apparatus may include an elongated, laterally flexible structure (e.g., 100) having first and second end portions (e.g., links 90a and 90b in FIG. 9) that are spaced from one another along the length of the elongated structure. Thus, for example, FIGS. 14 and 15 show that structure 100 is elongated, e.g., by extending lengthwise in a generally left-right direction across portions of these FIGS. Links 90a and 90b are at or near respective opposite ends of elongated structure 100. FIGS. like FIGS. 16 and 17 show that structure 100 is laterally flexible, by which it is meant that structure 100 can be curved transverse (or laterally) to the line along which it is shown extending longitudinally in FIGS. 14 and 15. Structure 100 is still an “elongated structure” even when thus curved or deflected laterally or transversely along its length. A first magnetic structure (e.g., 20a) may be secured to the first end portion (e.g., 90a; see, for example, FIG. 9, which shows the enlarged right-hand end of link 90a (shaped like a ball or a bead) captured in recess 80 in element 20a by the reduced interior size of the left-hand end portion of recess 80). A second magnetic structure (e.g., 20b) may be secured to the second end portion (e.g., 90b; see again FIG. 9, which shows the enlarged left-hand end of link 90b captured in element 20b in a manner similar to the capture of link 90a by element 20a). The first and second magnetic structures may be magnetically attracted to one another (e.g., by magnetic element 60a in structure 20a magnetically attracting magnetic element 60b in structure 20b) to hold the first and second end portions proximate to one another. Note in FIG. 10, for example, that when magnetic components 20a and 20b are adjacent to one another, they hold the links 90 that respectively extend off in opposite directions from those components 20a and 20b proximate to one another (i.e., in the effectively fixed relative positions shown in FIG. 10). Note further in FIG. 10, for example, that the remaining portions of elongated structure 100 extend continuously from the link 90 that connects to structure 20a all the way around tissue structure 200 to the link 90 that connects to structure 20b. Note still further that many of the FIGS. such as FIGS. 3-9 show the magnetic elements 60 in the magnetic structures 20, and how those magnetic elements 60 can be magnetically polarized so that they magnetically attract one another when brought together. As is conventional for such depiction, the magnetic pole of a magnetic element shown with a plus (“+”) sign magnetically attracts the oppositely magnetically polarized magnetic pole of another magnetic element shown with a minus (“−”) sign. Alternative terminology that can be used for polarization of magnets is that the “north” pole of one magnet magnetically attracts the “south” pole of another magnet. For example, in one common convention the “north” pole of a magnet is indicated by a plus sign and the “south” pole of the magnet is indicated by a minus sign.

In apparatus as recapitulated above, the elongated structure (e.g., 100) may extend away from the first magnetic structure (e.g., 20a) along a first axis (e.g., 50a). Similarly, the elongated structure (e.g., 100) may extend away from the second magnetic structure (e.g., 20b) along a second axis (e.g., 50b). In such apparatus, when the first and second magnetic structures hold the first and second end portions (e.g., 90a and 90b) proximate to one another, the first and second axes may be approximately aligned with one another (e.g., in FIG. 8 axes 50a and 50b are approximately aligned with one another). In this kind of context, “aligned” does not mean that the first and second axes are superimposed on one another. Indeed they typically are longitudinally offset or spaced from one another. However, “aligned” typically does mean that longitudinal extensions of axes 50a and 50b are at least approximately parallel, and they may in addition be coaxial or approximately coaxial (i.e., so that depicted axes 50a and 50b are two longitudinally spaced parts of one longer axis).

In apparatus as recapitulated above, when the first and second magnetic structures (e.g., 20a and 20b) hold the first and second end portions (e.g., 90a and 90b) proximate to one another, a primary direction of magnetic attraction between the first and second magnetic structures may be aligned with the first and second axes (e.g., 50a and 50b). Thus, for example, in embodiments like those shown in FIGS. 5-9, the primary force of magnetic attraction between magnetic elements 60a and 60b is parallel to axes 50a and 50b. To take just one more specific example of this, in FIG. 6 the “−” pole of magnetic element 60a magnetically attracts the “+” pole of magnetic element 60b primarily along a line that is parallel to axes 50a and 50b.

In apparatus as recapitulated above, when the first and second magnetic structures (e.g., 20a and 20b) hold the first and second end portions (e.g., 90a and 90b) proximate to one another, the first and second structures may mechanically interlock with one another. For example, in FIG. 8 the surfaces 74 interlock with surfaces 84. Similarly, in FIG. 9 the surface facing to the right from enlarged head 72 interlocks with the left-most free ends of fingers 82. In general, “interlock” or the like in this context means a form of mechanical retention that generally prevents separation of structures 20a and 20b and that cannot be undone without damage to the structure or without use of some other additional unlocking tool or apparatus.

In apparatus as recapitulated above, the interlock may take place as a result of motion of the first and second magnetic structures (e.g., 20a and 20b) toward one another along the primary direction. For example, in FIG. 8 the primary direction of magnetic attraction between elements 60a and 60b (and therefore between structures 20a and 20b) is parallel to axes 50a and 50b, and surfaces 74 and 84 interlock when structures 20a and 20b move into contact with one another parallel to axes 50a and 50b. FIG. 9 is similar in this respect (i.e., primary magnetic attraction between structures 20a and 20b is parallel to axes 50a and 50b, and elements 72 and 82 interlock with one another when structures 20a and 20b move into contact with one another along these axes).

In some apparatus such as is here being recapitulated, when the first and second magnetic structures (e.g., 20a and 20b) hold the first and second end portions (e.g., 90a and 90b) proximate to one another, the first and second magnetic structures may mechanically interengage with one another. For example, FIG. 7 shows an embodiment in which when magnetic structures 20a and 20b are in their final, face-to-face contact with one another, element 34a on structure 20a is received in recess 36b in structure 20b (and element 34b on structure 20b is received in recess 36a in structure 20a). Structures 20a and 20b are thereby “mechanically interengaged” with one another. In general, such “mechanically interengagement” provides at least some mechanical resistance to separation of structures 20a and 20b. However, such “mechanical interengagement” can typically be undone by some particular manipulation of structures 20a and 20b relative to one another. “Mechanical interengagement” may therefore be different from “mechanical interlocking” in such respects, for example, as (1) possibly being less permanent, and/or (2) possibly being more simply and easily reversed or undone (e.g., without the need for a special unlocking tool and/or without damage to the apparatus). Other examples of mechanical interengagement elements are features 34 in FIGS. 1-4.

In some apparatus such as in here being recapitulated, the first and second magnetic structures (e.g., 20a and 20b) may mechanically interengage with one another as a result of relative motion of the first and second magnetic structures transverse to the primary direction to make the first and second axes more nearly coaxial with one another. In FIG. 7, for example, element 20a can be moved to the right and then up to cause elements 34 to mechanically interengage with one another (i.e., by each one of elements 34 entering the recess 36 “behind” the other one of elements 34). When this is done, axes 50a and 50b become more nearly coaxial with one another. The movement described immediately above as “up” is an example of relative motion transverse to axes 50a and 50b.

In some apparatus as has just been recapitulated, the first and second magnetic elements (e.g., 20a and 20b) may include mechanical interlock elements (e.g., 600, 610, 620, 622; 600′, 602′, 610′, 620′, 622′) for mechanically interlocking the first and second magnetic structures to one another after a predetermined amount of said relative motion (i.e., relative motion transverse to axes 50a and 50b).

In some apparatus as is here being recapitulated, when the first and second magnetic structures (e.g., 20a and 20b) hold the first and second end portions (e.g., 90a and 90b) proximate to one another, the primary direction of magnetic attraction between the first and second magnetic structures may be transverse to the first and second axes (e.g., 50a and 50b). For example, in embodiments like those shown in FIGS. 1-4, the primary direction of magnetic attraction between magnetic elements 60a and 60b is transverse (in this case perpendicular) to axes 50a and 50b. More particularly, this primary direction is along an axis (e.g., 40) that extends from the “+” pole of magnetic element 60b to the “−” pole of magnetic element 60a.

In some apparatus as has just been recapitulated, when the first and second magnetic structures (e.g., 20a and 20b) hold the first and second end portions (e.g., 90a and 90b) proximate to one another, the first and second magnetic structures may mechanically interengage with one another. For example, elements 34 in FIGS. 1-4 may provide such mechanical interengagement between structures 20a and 20b when those structures are fully together. “Mechanical interengagement” here has meaning similar to that described earlier for this term.

In some apparatus as has just been recapitulated the first and second magnetic structures (e.g., 20a and 20b) may mechanically interengage with one another by interengaging elements (e.g., 34) that mechanically resist separation of the first and second magnetic structures parallel to the first and second axes (e.g., 50a and 50b). For example, in any of FIGS. 1-4, when the elements 34 on one of structures 20 mechanically interengage with the elements 34 on the other of structures 20, that mechanical interengagement mechanically resists separation of structures 20a and 20b parallel to axes 50a and 50b.

Further recapitulating and/or extending the foregoing, certain aspects of the disclosure relate to methods of implanting in a patient's body (e.g., 300) apparatus that includes an elongated, laterally flexible structure (e.g., 100) having first and second end portions (e.g., 90b and 90a) that are spaced from one another along a length of the elongated structure, and first and second magnetic structures (e.g., 20b and 20a) that are respectively secured to the first and second end portions (e.g., as shown in FIG. 9). Such methods may include engaging the first magnetic structure (e.g., with instrument 400 via loop 120b) to manipulate position of the first magnetic structure in the patient's body (e.g., as shown in various stages in FIGS. 14, 15, 16, etc.). Such methods may further include engaging the second magnetic structure (e.g., with instrument 500 via loop 120a) to manipulate position of the second magnetic structure in the patient's body (e.g., as shown in various stages in FIGS. 16, 17, 18, etc.). Such methods may still further include using the engaging of the first and second magnetic structures to position the first and second magnetic structures relative to one another (e.g., as shown in various stages in FIGS. 14-20) so that the first and second magnetic structures magnetically attract one another and hold the first and second end portions proximate to one another (e.g., as shown in any of FIGS. 10 and 20-22).

In at least some methods as recapitulated above, the “using” may include wrapping the elongated structure (e.g., 100) around a tissue structure (e.g., 200) in the patient's body (e.g., as shown in various stages in FIGS. 13-17) prior to positioning the first and second magnetic structures (e.g., 20b and 20a) so that the first and second magnetic structures magnetically attract one another (e.g., as in FIGS. 18-20) and hold the first and second end portions (e.g., 90b and 90a) proximate to one another (e.g., as in any of FIGS. 10 and 20-22).

In some methods as recapitulated above, the tissue structure may comprise the patient's esophagus (e.g., 200).

Methods as recapitulated above may further include, after the above-mentioned “using,” releasing the engaging of the first and second magnetic structures (e.g., as shown in FIG. 21 wherein instrument 400 no longer engages loop 120b and instrument 500 no longer engages loop 120a).

In some methods as recapitulated above, laparoscopic instrumentation (e.g., 400 and 500) may be used to perform at least some of the above-mentioned “engaging.”

It will be understood that the foregoing is only illustrative of the principles of the disclosure, and that various modifications can be made by those skilled in the art without departing from the scope and spirit of the disclosure. For example, other forms of mechanical reinforcement for magnetically assisted clasps 10 will occur to those skilled in the art.

What is claimed is:

1. Apparatus for implanting in a patient's body comprising:
an elongated, laterally flexible structure having first and second end portions that are spaced from one another along a length of the elongated structure;
a first magnetic structure secured to the first end portion; and
a second magnetic structure secured to the second end portion, the first and second magnetic structures being magnetically attracted to one another to hold the first and second end portions proximate to one another.

2. The apparatus defined in claim 1 wherein the elongated structure extends away from the first magnetic structure along a first axis, wherein the elongated structure extends away from the second magnetic structure along a second axis, and wherein when the first and second magnetic structures hold the first and second end portions proximate to one another, the first and second axes are approximately aligned with one another.

3. The apparatus defined in claim 2 wherein when the first and second magnetic structures hold the first and second end portions proximate to one another, a primary direction of magnetic attraction between the first and second magnetic structures is aligned with the first and second axes.

4. The apparatus defined in claim 3 wherein when the first and second magnetic structures hold the first and second end portions proximate to one another, the first and second magnetic structures mechanically interlock with one another.

5. The apparatus defined in claim 4 wherein the interlock takes place as a result of motion of the first and second magnetic structures toward one another along the primary direction.

6. The apparatus defined in claim 3 wherein when the first and second magnetic structures hold the first and second end portions proximate to one another, the first and second magnetic structures mechanically interengage with one another.

7. The apparatus defined in claim 6 wherein the first and second magnetic structures mechanically interengage with one another as a result of relative motion of the first and second magnetic structures transverse to the primary direction to make the first and second axes more nearly coaxial with one another.

8. The apparatus defined in claim 7 wherein the first and second magnetic structures include mechanical interlock elements for mechanically interlocking the first and second magnetic structures to one another after a predetermined amount of said relative motion.

9. The apparatus defined in claim 2 wherein when the first and second magnetic structures hold the first and second end portions proximate to one another, a primary direction of magnetic attraction between the first and second magnetic structures is transverse to the first and second axes.

10. The apparatus defined in claim 9 wherein when the first and second magnetic structures hold the first and second end portions proximate to one another, the first and second magnetic structures mechanically interengage with one another.

11. The apparatus defined in claim 10 wherein the first and second magnetic structures mechanically interengage with one another by interengaging elements that mechanically resist separation of the first and second magnetic structures parallel to the first and second axes.

12. A method of implanting in a patient's body apparatus that includes an elongated, laterally flexible structure having first and second end portions that are spaced from one another along a length of the elongated structure, and first and second magnetic structures that are respectively secured to the first and second end portions, the method comprising:
engaging the first magnetic structure to manipulate position of the first magnetic structure in the patient's body;
engaging the second magnetic structure to manipulate position of the second magnetic structure in the patient's body;
using the engaging of the first and second magnetic structures to position the first and second magnetic structures relative to one another so that the first and second magnetic structures magnetically attract one another and hold the first and second end portions proximate to one another.

13. The method defined in claim 12 wherein the using comprises:
wrapping the elongated structure around a tissue structure in the patient's body prior to positioning the first and second magnetic structures so that the first and second magnetic structures magnetically attract one another and hold the first and second end portions proximate to one another.

14. The method defined in claim 12 wherein the tissue structure comprises the patient's esophagus.

15. The method defined in claim 12 further comprising:
after the using, releasing the engaging of the first and second magnetic structures.

16. The method defined in claim 12 further comprising:
using laparoscopic instrumentation to perform at least some of the engaging.
